(19) United States
(12) Patent Application Publication
(10) Pub. No.: US 2014/0248626 A1
(43) Pub. Date: Sep. 4, 2014

(54) METHODS FOR REMOVING NUCLEIC ACID CONTAMINATION FROM REAGENTS

(71) Applicant: LIFE TECHNOLOGIES CORPORATION, Carlsbad, CA (US)

(72) Inventors: Jason Yingjie LIU, Foster City, CA (US); Elena Bolchakova, Union City, CA (US); Jaiprakash Shewale, Santa Clara, CA (US); Manohar Furtado, San Ramon, CA (US)

(73) Assignee: LIFE TECHNOLOGIES CORPORATION, Carlsbad, CA (US)

(21) Appl. No.: 14/192,631

(22) Filed: Feb. 27, 2014

Related U.S. Application Data
(63) Continuation of application No. 12/994,145, filed on Apr. 22, 2011, now abandoned.
(60) Provisional application No. 61/055,864, filed on May 23, 2008.

(51) Int. Cl. C12Q 1/68

(52) U.S. Cl. CPC C12Q 1/6806
USPC 435/6.12; 435/262

(57) ABSTRACT

In general, the disclosed method can be used to remove contaminating microbes and nucleic acids from microorganisms-derived reagents, apparatus and processes (materials and apparatus) related to PCR (and RT-PCR), including sample prep reagents and materials that are used to isolate, purify and detect nucleic acids.

### FIELD

In general, the disclosed invention relates to the decontamination of DNA or RNA contaminated reagents used for the analysis of nucleic acid in reactions such as a nucleic acid amplification reaction or a Sanger sequencing reaction.

### BACKGROUND

The polymerase chain reaction (PCR) is a method that allows exponential amplification of nucleic acid sequences within a longer double-stranded nucleic acid molecule. The nucleic acid can be either DNA or RNA. PCR can amplifies and detects RNA by first using a reverse transcriptase enzyme to convert RNA into complementary DNA (cDNA) which is then amplified by PCR. Real-time PCR was developed for the purpose of quantitative detection of target nucleic acid molecules. One of the ways to monitor the PCR amplification process is by adding fluorescent dyes that are specific for double-stranded DNA to the PCR reaction mix or using labeled probes in a real-time PCR reaction. Fluorescent intensity doubles after each thermal cycle during the logarithmic amplification phase of a PCR reaction. SYBR® Green dye is one example of a dye that binds to double-stranded DNA but not to single-stranded DNA and is frequently used in real-time PCR reactions.

PCR is an extremely sensitive technology, which is capable of detecting as little as a single target nucleic acid molecule. However, in order to achieve single molecule detection sensitivity and specificity, the reagents, materials and apparatus used in PCR should be free of contaminating nucleic acids. The components of a typical PCR master mix reagent mixture includes, but are not limited to at least a thermalstable DNA polymerase, dNTPs, salt, and optionally, a reverse transcriptase, a fluorescent dye(s) and various additives. Oligonucleotide primers (and probes) are not incorporated into the PCR master mix, but added to the PCR reaction mixture prior to performing PCR. None of the typical PCR master mix components offered commercially can be obtained free of bacterial/microbes and/or free of bacterial nucleic acid. Indeed, C_{T} values for no template control (NTC) have been observed for PCR reactions with bacterial targets.

The enzyme components used in PCR reactions are commonly prepared by recombinant DNA methodologies. The polymerase enzymes used have bacterial origins. Therefore, the PCR master mix reaction components as obtained from commercial vendors are never completely assured to be either microbe- or microbial nucleic acid-free. Since the PCR master mix comprises diverse components in terms of biological and chemical properties, filtration as a method of removing nucleic acid contaminants can alter one or more components of the PCR reaction mix, resulting in reduced PCR efficiency. Purification and removal of contaminating nucleic acids from individual PCR components is feasible, but several technologies such as size exclusion, ionic and affinity filtration need to be developed and optimized for each type of component category. This increases the complexity and cost of the manufacture processes.

Nucleic acid amplification, isolation or purification is the objective when working with DNA or RNA. The presence of residual nucleases following nuclease treatment could destroy the target nucleic acid template resulting in reduced yields of isolated nucleic acid, failure to amplify or detect target nucleic acid sequences and degraded isolated nucleic acids. The purity and stability of target nucleic acid is also a serious concern when using nucleic acids for diagnostic or forensic applications where sample size is very limited. The presence of contaminating nucleic acids that degrade the target nucleic acid sample can render the analysis useless. Additionally, biopharmaceutical manufacturing mandates minimum amounts of residual nucleic acids or microbes as a consequence of the manufacturing process. Contaminating microbes and their nucleic acids can preclude an accurate assessment of residual microbe and nucleic acid levels. Thus, there exists a need in the art to remove contaminating nucleic acids from molecular biology and biopharmaceutical reagents and apparatus used in research, manufacturing, diagnostic, forensic, nucleic acid isolation, and purification methods.

Therefore, it is extremely challenging to build a PCR master mix reagent kit free of microbial cells and nucleic acids.

### SUMMARY OF SOME EMBODIMENTS OF THE INVENTION

In one embodiment of the current teachings includes, a method for removing nucleic acids (e.g., contaminating microbial nucleic acids) from master mix reagents and components thereof. The method uses a nuclease, either a DNase or an RNase added to a component of the master mix or to the master mix itself (minus primers and probe(s)). The nuclease may be added as a solution or bound to an insoluble matrix or a solid support, such as a bead selected from a group consisting of magnetic, non-magnetic, glass, and cellulose beads. The immobilized nuclease can be removed by filtration, centrifugation or magnetic separation. Alternatively, the nuclease is inactivated by heat following incubation.

The current teachings also provide methods for making a real-time PCR reagent kit for the detection of trace amounts of microbial contaminants in reagents or pharmaceutical raw materials and finished products. To illustrate, all the reagents in the testing kit can be substantially free of contaminating microorganisms and microbial nucleic acids. The present teaching provides an effective, simple to implement method of removing contaminating microbes and nucleic acids simultaneously form a PCR master mix or similar reagent containing components having different chemical properties.

The present teachings also provide methods for removing nucleic acid contamination in a PCR master mix reagent by a) adding a nuclease to the PCR master mix reagent, b) incubating the PCR master mix reagent containing nuclease to digest the contaminating nucleic acid (e.g., DNA) at an effective temperature for a sufficient period of time, and c) inactivating the nuclease's activity. The nuclease acts to hydrolyze DNA or RNA molecules, if present, in the reagent components comprising the PCR master mix.

The present teachings further provide for the use of nuclease immobilized on beads in some embodiments of the subject methods. The beads are selected from a group consisting of magnetic and non-magnetic beads. The nuclease-bead complex can be separated from the reagents after the completion of the nuclease reaction by centrifugation, filtration or magnetic separation.

The present teachings further comprise a method, for removing microorganism nucleic acid contamination in a PCR master mix comprising a) passing the PCR master mix through a column packed with immobilized nuclease-coated beads or a tube internally coated with a nuclease at an optimized flow rate and temperature to digest the contaminating nucleic acids and b) collect the nuclease treated PCR master mix.

In another embodiment, the current teachings are also applicable to a method for removing microbial RNA contamination in a PCR master mix by a) exposing PCR master mix to RNase immobilized on solid support, b) incubating the PCR master mix reagent with the immobilized RNase to digest RNA at an effective temperature for sufficient time, and c) remove immobilized RNase from the PCR master mix. The RNase (ribonuclease) enzymes act by either phosphorylation or hydrolysis. This method is also applicable for the removal of contaminating DNA by use of a DNase enzyme.

In another embodiment, the present teachings provide methods for removing nucleic acid contamination from a reagent component or a reagent mixture by adding a plurality of nucleases to the component or reagent mixture, incubating the resulting mixture and then removing or inactivating the plurality of nucleases. The plurality of nucleases may be added as a solution, bound to an insoluble matrix or a solid support or a combination thereof. The solid support can be a bead selected from a group consisting of magnetic, non-magnetic, glass, and cellulose beads. The immobilized nuclease(s) can be removed by filtration, centrifugation or magnetic separation. Alternatively, the nuclease(s) can be inactivated by heat following incubation. The plurality of nucleases can be at least two DNases, at least two RNases, or a combination of at least one DNase and at least one RNase.

In another embodiment, the present teachings provide a nuclease-free reagent, PCR master mix, a component of a PCR master mix or an apparatus used in the isolation of a nucleic acid produced by a) adding a nuclease to a contaminated reagent or apparatus, b) incubating the contaminated reagent or apparatus to digest the contaminating nucleic acid (e.g., DNA or RNA) at an effective temperature for a sufficient period of time, and c) inactivating the nuclease's activity. The process uses a nuclease, either a DNase or an RNase added to the contaminated reagent or apparatus. The nuclease may be added as a solution or bound to an insoluble matrix or a solid support, such as a bead selected from a group consisting of magnetic, non-magnetic, glass, and cellulose beads. The immobilized nuclease can be removed by filtration, centrifugation or magnetic separation. Alternatively, the nuclease is inactivated by heat following incubation.

### DRAWINGS

The skilled artisan will understand that the drawings described below are for illustration purposes only. The drawings are not intended to limit the scope of the present teachings in any way.

FIG. 1. TURBO DNase™ enzyme treatment of a PCR master mix spiked with DNA. DNase treatment was carried out at 37° C. for (a) 0 min., (b) 10 min., (c) 20 (d) 30 min., and (e) 40 min. DNase was inactivated by a heat treatment at 75° C. for 10 min.

FIG. 2. TURBO DNase™ enzyme treatment of PCR master mix containing DNA. TURBO DNase™ enzyme was first heated at 75° C. for 10 min. Subsequently, DNase treatment was carried out at 37° C. for (a) 0 min., (b) 10 min., (c) 20 min., (d) 30 min., and (e) 40 min. The DNase was then inactivated at 75° C. for 10 min.

FIG. 3. PCR detection of contaminating E. coli DNA using DNase treated (right) and untreated (left) PCR reaction mix. Test samples were spiked with E. coli DNA at (a) 100 copies, (b) 10 copies, (c) 1 copy and (d) no template control (NTC).

FIG. 4. Dissociation curves for 1 copy E. coli (c) and NTC (d) with TURBO DNase™ enzyme.

### DESCRIPTION

For the purposes of interpreting of this specification, the following definitions will apply and whenever appropriate, terms used in the singular will also include the plural and vice versa. In the event that any definition set forth below conflicts with the usage of that word in any other document, including any document incorporated herein by reference, the definition set forth below shall always control for purposes of interpreting this specification and its associated claims unless a contrary meaning is clearly intended (for example in the document where the term is originally used). It is noted that, as used in this specification and the appended claims, the singular forms “a,” “an,” and “the,” include plural referents unless expressly and unequivocally limited to one referent. The use of “or” means “and/or” unless stated otherwise. The use of “comprise,” “comprises,” “comprising,” “include,” “includes,” and “including” are interchangeable and not intended to be limiting. Furthermore, where the description of one or more embodiments uses the term “comprising,” those skilled in the art would understand that, in some specific instances, the embodiment or embodiments can be alternatively described using the language “consisting essentially of” and/or “consisting of”.

As used herein, “DNA” refers to deoxyribonucleic acid in its various forms as understood in the art, such as genomic DNA, cDNA, isolated nucleic acid molecules, vector DNA, and chromosomal DNA. “Nucleic acid” refers to DNA or RNA in any form. Examples of isolated nucleic acid molecules include, but are not limited to, recombinant DNA molecules contained in a vector, recombinant DNA molecules maintained in a heterologous host cell, partially or substantially purified nucleic acid molecules, and synthetic DNA molecules. Typically, an “isolated” nucleic acid is free of sequences which naturally flank the nucleic acid (i.e., sequences located at the 5′ and 3′ ends of the nucleic acid) in the genomic DNA of the organism from which the nucleic acid is derived. Moreover, an “isolated” nucleic acid molecule, such as a cDNA molecule, is generally substantially free of other cellular material or culture medium when produced by recombinant techniques, or free of chemical precursors or other chemicals when chemically synthesized.

As used herein, “incubating” refers to maintaining a state of controlled conditions, e.g., temperature, over a period of time.

As used herein, “DNA-digesting enzyme” refers to a nuclease that degrades double- and single-stranded DNA into individual nucleotides or fragments to small to interfere to any significance in the desired reactions. For example, DNase (deoxyribonuclease) e.g., DNase I functions by hydrolyzing phosphodiester linkages of DNA often at phosphodiester linkages in proximity to a pyrimidine nucleotide, resulting in a 5′-phosphate terminated polynucleotide with a free hydroxyl group at the 3′ position.

As used herein, “DNase” refers to any enzyme which degrades DNA. DNase enzymes can be an endonuclease which cuts within a polynucleotide chain, e.g., a restriction enzyme, an exonuclease which utilizes a free end of a polynucleotide in order to degrade a DNA molecule. DNA-digesting enzymes can be inactivated by heat, at a temperature of at least 75° C.

As used herein, “RNase” refers to an enzyme which hydrolyses RNA and can be single-strand specific, e.g., RNase T1, and double-strand specific, e.g., RNase III.

As used herein, TURBO DNase™ enzyme (Ambion, Austin, Tex.) refers to a DNase enzyme developed using a protein engineering approach that introduced amino acid changes into the DNA binding pocket of wild-type DNase I. TURBO DNase™ enzyme has a greater affinity than wild-type DNase I for DNA and can digest DNA into fragments even when the DNA concentration is in the nanomolar (nM) range.

As used herein, “nuclease” refers to an enzyme capable of cleaving the phosphodiester bonds between the nucleotide subunits of nucleic acids. The term “nuclease-free” refers to a nuclease having no activity or very low nuclease activity.

As used herein, “microbial” or “microorganisms” includes but is not limited to bacteria, fungi, and yeast and includes all other microbial and biological species.

As used herein, “PCR master mix” refers to a composition whose components include, but are not limited to, buffers and salt as well as polymerase enzyme(s) that are used for DNA amplification using the polymerase chain reaction (PCR). The PCR master mix referred to herein does not include primers and probes that may be necessary for carrying out PCR amplification or detection of amplified products.

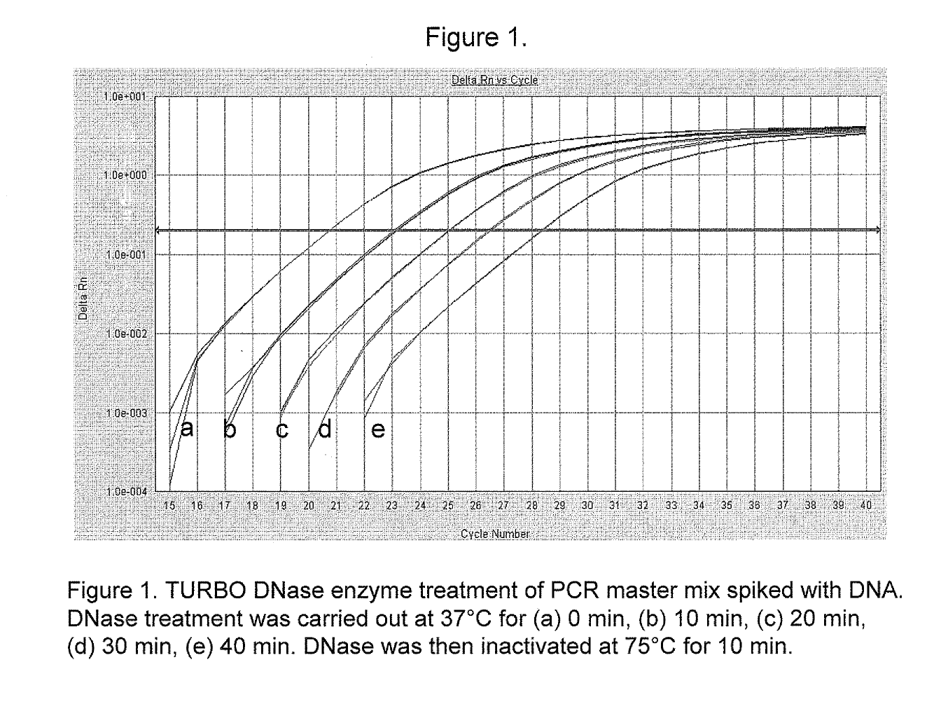

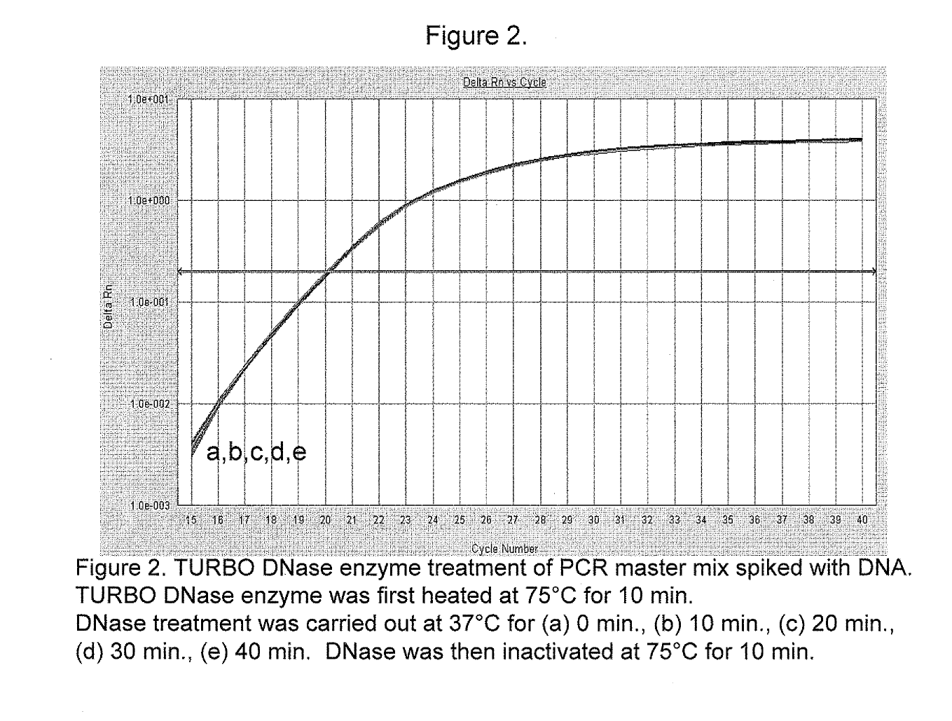

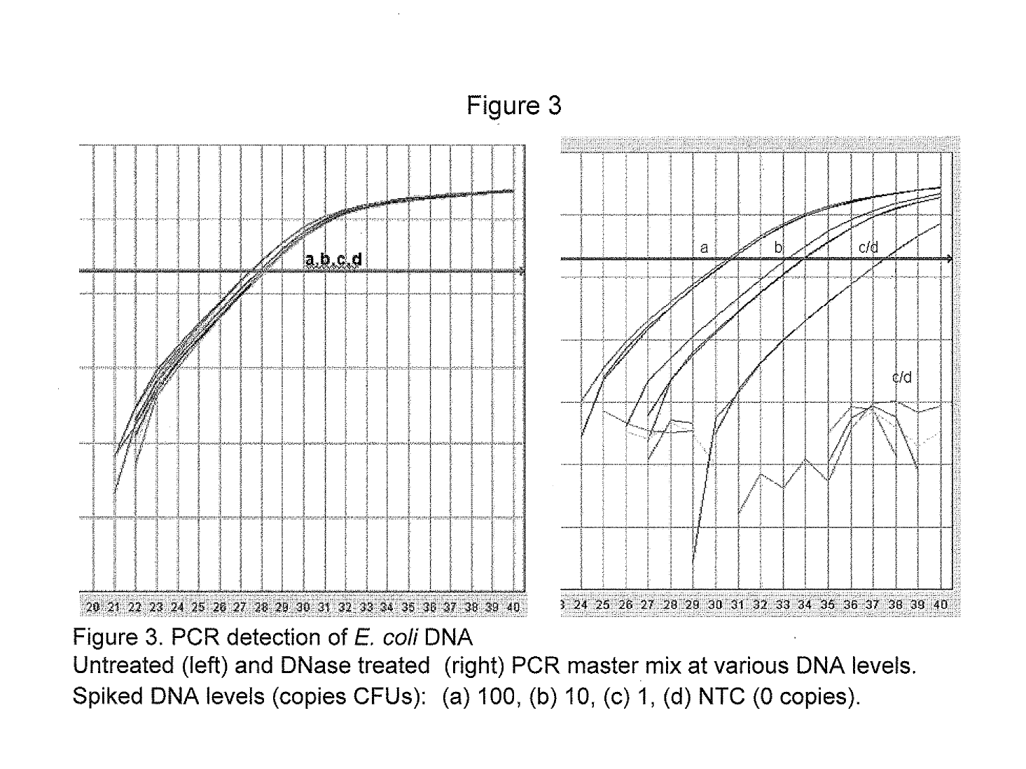

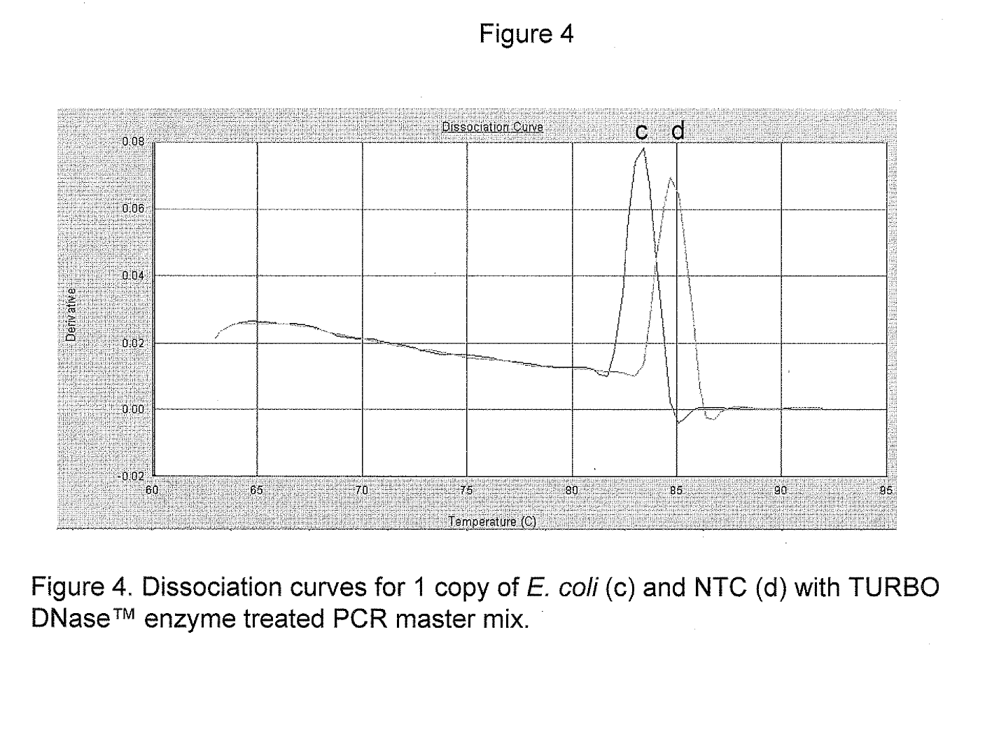

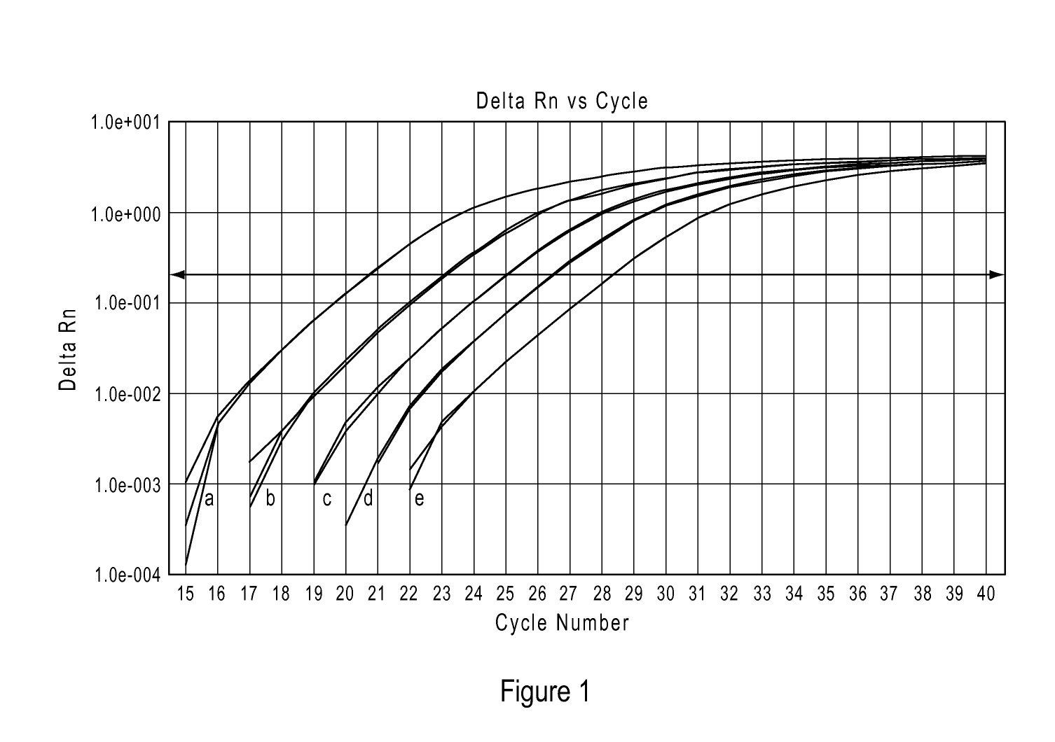

As used herein, “inactivation” or “inactivate” refers to the destruction of the catalytic activity of an enzyme such that the function of the enzyme is rendered non-functional. Various means by which an enzyme is inactivated include, but are not limited to, denaturation techniques such as heat, chemical or irradiation methodologies. Nuclease inactivation can also include the removal of an enzyme from a reaction vessel in which enzymatic activity occurred. As shown in FIG. 1, enzymatic activity is approximately at least 60% decreased after 10 minutes at 75° C. and substantially inactive after 20 minutes.

As used herein, beads refer to either magnetic beads or non-magnetic beads made of various materials to which a DNase or RNase protein can be bound by physical or chemical means.

As used herein, “apparatus” refers to test tubes, microfuge tubes, pipets, pipet tips and materials that come into contact with nucleic acids due the analysis, isolation and purification of nucleic acids.

As used herein, the “polymerase chain reaction” or PCR is a an amplification of nucleic acid consisting of an initial denaturation step which separates the strands of a double stranded nucleic acid sample, followed by repetition of (i) an annealing step, which allows amplification primers to anneal specifically to positions flanking a target sequence; (ii) an extension step which extends the primers in a 5′ to 3′ direction thereby forming an amplicon polynucleotide complementary to the target sequence, and (iii) a denaturation step which causes the separation of the amplicon from the target sequence (Mullis et al., eds, The Polymerase Chain Reaction, BirkHauser, Boston, Mass. (1994). Each of the above steps may be conducted at a different temperature, preferably using an automated thermocycler (Applied Biosystems LLC, a division of Life Technologies Corporation, Foster City, Calif.). If desired, RNA samples can be converted to DNA/RNA heteroduplexes or to duplex cDNA by methods known to one of skill in the art. The PCR method also includes reverse transcriptase-PCR and other reactions that follow principles of PCR.

As used herein, “amplifying” and “amplification” refers to a broad range of techniques for increasing polynucleotide sequences, either linearly or exponentially. Exemplary amplification techniques include, but are not limited to, PCR or any other method employing a primer extension step. Other nonlimiting examples of amplification include, but are not limited to, ligase detection reaction (LDR) and ligase chain reaction (LCR). Amplification methods may comprise thermal-cycling or may be performed isothermally. In various embodiments, the term “amplification product” includes products from any number of cycles of amplification reactions.

In certain embodiments, amplification methods comprise at least one cycle of amplification, for example, but not limited to, the sequential procedures of: hybridizing primers to primer-specific portions of target sequence or amplification products from any number of cycles of an amplification reaction; synthesizing a strand of nucleotides in a template-dependent manner using a polymerase; and denaturing the newly-formed nucleic acid duplex to separate the strands. The cycle may or may not be repeated.

There are many known methods of amplifying nucleic acid sequences including e.g., PCR. See, e.g., PCR Technology: Principles and Applications for DNA Amplification (ed. H. A. Erlich, Freeman Press, NY, N.Y., 1992); PCR Protocols: A Guide to Methods and Applications (eds. Innis, et al., Academic Press, San Diego, Calif., 1990); Mattila et al., Nucleic Acids Res. 19, 4967 (1991); Eckert et al., PCR Methods and Applications 1, 17 (1991); PCR (eds. McPherson et al., IRL Press, Oxford); and U.S. Pat. Nos. 4,683,202, 4,683,195, 4,800,159 4,965,188 and 5,333,675 each of which is incorporated herein by reference in their entireties for all purposes.

Nucleic acid amplification techniques are traditionally classified according to the temperature requirements of the amplification process. Isothermal amplifications are conducted at a constant temperature, in contrast to amplifications that require cycling between high and low temperatures. Examples of isothermal amplification techniques are: Strand Displacement Amplification (SDA; Walker et al., 1992, Proc. Natl. Acad. Sci. USA 89:392 396; Walker et al., 1992, Nuc. Acids. Res. 20:1691 1696; and EP 0 497 272, all of which are incorporated herein by reference), self-sustained sequence replication (3SR; Guatelli et al., 1990, Proc. Natl. Acad. Sci. USA 87:1874 1878), the Q.beta. replicase system (Lizardi et al., 1988, BioTechnology 6:1197 1202), and the techniques disclosed in WO 90/10064 and WO 91/03573.

Examples of amplification techniques that require temperature cycling are: polymerase chain reaction (PCR; Saiki et al., 1985, Science 230:1350 1354), ligase chain reaction (LCR; Wu et al., 1989, Genomics 4:560 569; Barringer et al., 1990, Gene 89:117 122; Barany, 1991, Proc. Natl. Acad. Sci. USA 88:189 193), transcription-based amplification (Kwoh et al., 1989, Proc. Natl. Acad. Sci. USA 86:1173 1177) and restriction amplification (U.S. Pat. No. 5,102,784).

Other exemplary techniques include Nucleic Acid Sequence-Based Amplification (“NASBA”; see U.S. Pat. No. 5,130,238), Q.beta. replicase system (see Lizardi et al., BioTechnology 6:1197 (1988)), and Rolling Circle Amplification (see Lizardi et al., Nat Genet. 19:225 232 (1998)). The amplification primers of the present invention may be used to carry out, for example, but not limited to, PCR, SDA or tSDA. Any of the amplification techniques and methods disclosed herein can be used to practice the claimed invention as would be understood by one of ordinary skill in the art.

The words “preferred” and “preferably” refer to embodiments of the invention that may afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the invention.

The terms “comprises” and variations thereof do not have a limiting meaning where these terms appear in the description and claims. Unless otherwise specified, “a,” “an,” “the,” and “at least one” are used interchangeably and mean one or more than one.

Also herein, the recitations of numerical ranges by endpoints include all numbers subsumed within that range (e.g., 1 to 5 includes 1, 1.5, 2, 2.75, 3, 3.80, 4, 5, etc.). The term “and/or” means one or all of the listed elements or a combination of any two or more of the listed element.

The reagents used in the isolation, purification and analysis of target RNA or DNA nucleic acids are not without trace amounts of contaminating nucleic acids and/or microbial cells from the bacteria used to produce the reagents, including, but not limited to, polymerases, nucleases and the like. Table 1 lists possible components of a PCR master mix. Not one component listed is either microbial cell or nucleic acid free.

TABLE 1

| Component | Trace Contaminants |
|---|---|
| Water | Microbial DNA, RNA, microbial cells |
| DMSO | Microbial DNA, RNA, microbial cells |
| SYBR ® Green dye | Microbial DNA, RNA, microbial cells |
| Tris•HCl | Microbial DNA, RNA, microbial cells |
| Tween-20 | Microbial DNA, RNA, microbial cells |
| ROX dye | Microbial DNA, RNA, microbial cells |
| Gelatin | Microbial DNA, RNA, microbial cells |
| Magnesium chloride | Microbial DNA, RNA, microbial cells |
| dNTP Mix w/dUTP | Microbial DNA, RNA, microbial cells |
| dNTP Mix w/dTTP | Microbial DNA, RNA, microbial cells |
| Glycerol | Microbial DNA, RNA, microbial cells |
| TMAC | Microbial DNA, RNA, microbial cells |
| CHAPS | Microbial DNA, RNA, microbial cells |
| Sodium azide | Microbial DNA, RNA, microbial cells |
| DNA polymerase | Microbial DNA, RNA, microbial cells |

Preparing nucleic acid free reagents using nuclease enzymes is counterintuitive because the very nuclease used to degrade the trace contaminating nucleic acids and proteins can also degrade the target RNA or DNA used for analysis in molecular biology methods such as nucleic acid isolation, PCR, nucleic acid purification (removing contaminating DNA from RNA isolation, and contaminating RNA from DNA isolation), and other methods (see U.S. Pat. No. 7,067,298). Therefore, the addition of nuclease to reagents thus will ultimately be used with polynucleotides is are contrary to standard practices of DNA and RNA methodologies, biopharmaceutical manufacturing, diagnostic and forensic practices and enzyme production methods. Unexpectedly, the use of nucleases, including, but not limited to, e.g., TURBO DNase™ enzyme, eliminates detectable nucleic acid contaminants and following heat inactivation of the nuclease, does not degrade the target nucleic acid of interest. Thus, DNA-free, nucleic acid free and microbial free reagents (either as individual components or in combinations, including, but not limited to, a PCR master mix, a sequencing reaction mix and a genotyping cocktail), and apparatus are obtainable using the claimed invention.

The prototype PCR master mix that has been formulated using the components listed in Table 1 provides a very low C_{T} (about 26) even when no target DNA was present in the sample (NTC). This indicates the presence of either microbial cells or microbial nucleic acids in the PCR master mix. Since none of the components are certified microbial or nucleic acid free (free of cells and/or nucleic acids) by the suppliers, potentially each of the components may contain trace amount of microbial cells or microbial nucleic acids.

In one aspect, the PCR master mix reagent containing microbial contamination is treated with a DNA-digesting enzyme by itself or in the presence of at least one of Mn²⁺, Ca²⁺ and Mg²⁺ ions. The choice of divalent cation and concentration is adjustable based on the PCR master mix composition and the type of DNA-digesting enzyme used. DNA-digesting enzymes are known to one of skill in the art and included but are not limited to both natural, synthetic and chemically modified deoxyribonuclease enzymes (DNase enzyme).

Nucleases are either DNases or RNases. Nucleases can be isolated from most organisms and may be prepared using recombinant techniques known to one of skill in the art. DNases degrade DNA and RNases degrade RNA. The method of action and reaction conditions for each varies and selection is dependent upon the desired result as would be known to one of skill in the art. There are two types of DNases, DNase I and DNase II. DNase I has a pH optimum near neutral and an obligatory requirement for divalent cations, and creates free 5′-phosphate deoxynucleotide products. DNase II has an acid pH optimum, can be activated by divalent cations, and produces a free 3′-phosphate deoxynucleotides upon hydrolysis of DNA. Reagents used for PCR would be treated by a DNase I, as would be understood by one of skill in the art.

DNase I enzymes have historically been prepared from bovine pancreas, one of the richest sources of RNase activity. Therefore, it is often hard to obtain DNase I sufficiently free of RNase that it will not compromise RNA analysis experiments. Recombinant DNase I (rDNase I) is preferred if the PCR reagent mix is intended for use in a reverse-transcription PCR application. This is because rDNase is usually prepared in a host that has RNase levels that are 1×10⁷ fold lower than bovine pancreas. Some of the commercially available recombinant DNase I are rDNase I from Sigma (P/N AMPD1); from Invitrogen (P/N 18068015); from Roche (P/N 04716728001); from Ambion (P/N AM2235) and TURBO DNase™ (P/N AM2238).

TURBO DNase™ was developed using a protein engineering approach that introduced amino acid changes into the DNA binding pocket of wild-type DNase I. These changes markedly increase the affinity of the protein for DNA. The result is a versatile enzyme that has a 6-fold lower Kₘ for DNA, and an ability to maintain at least 50% of peak activity in solutions approaching 200 mM monovalent salt, even when the DNA concentration is in the nanomolar (nM) range. Therefore, DNase I, and in particular TURBO DNase™ enzyme, can be used to digest any DNA contaminant resulting from reagent manufacture, especially reagents for PCR, DNA isolation and apparatus used in the process of DNA purification and isolation.

RNase is a nuclease that catalyzes the degradation of RNA into smaller components. RNase can be divided into endoribonucleases and exoribonucleases, and comprise several sub-classes within the EC 2.7 (for the phosphorolytic enzymes) and EC 3.1 (for the hydrolytic enzymes) classes of enzymes. Major types RNases are RNase A (cleaves 3′ end of unpaired C and U residues, leaving a 3′-phosphorylated product, via a monophosphate), RNase H is a ribonuclease that cleaves the RNA in a DNA/RNA duplex to produce ssDNA. RNase H is a non-specific endoribonuclease and catalyzes the cleavage of RNA via a hydrolytic mechanism, aided by an enzyme-bound divalent metal ion (leaving a 5′-phosphorylated product). RNase I cleaves at the 3′-end of ssRNA and at all dinucleotide bonds (leaving a 5′ hydroxyl, and 3′ phosphate, via a 2′,3′-cyclic monophosphate intermediate). RNase II is responsible for the processive 3′-to-5′ degradation of single-stranded RNA. RNase T1 is sequence specific for single-stranded RNAs. It cleaves at the 3′-end of unpaired G residues. RNase V1 is non-sequence specific for double-stranded RNAs. It cleaves base-paired nucleotide residues. Some of the commercially available RNase; are RNase A (P/N AM2274), RNase T1(P/N AM2283) and RNase V1 (P/N AM2275) from Ambion. RNase A (P/N12091021) from Invitrogen.

As used herein, nuclease refers to either a DNase enzyme or an RNase enzyme. Thus, methods described for DNase can equally be applied to use of an RNase enzyme as would be understood by one of skill in the art.

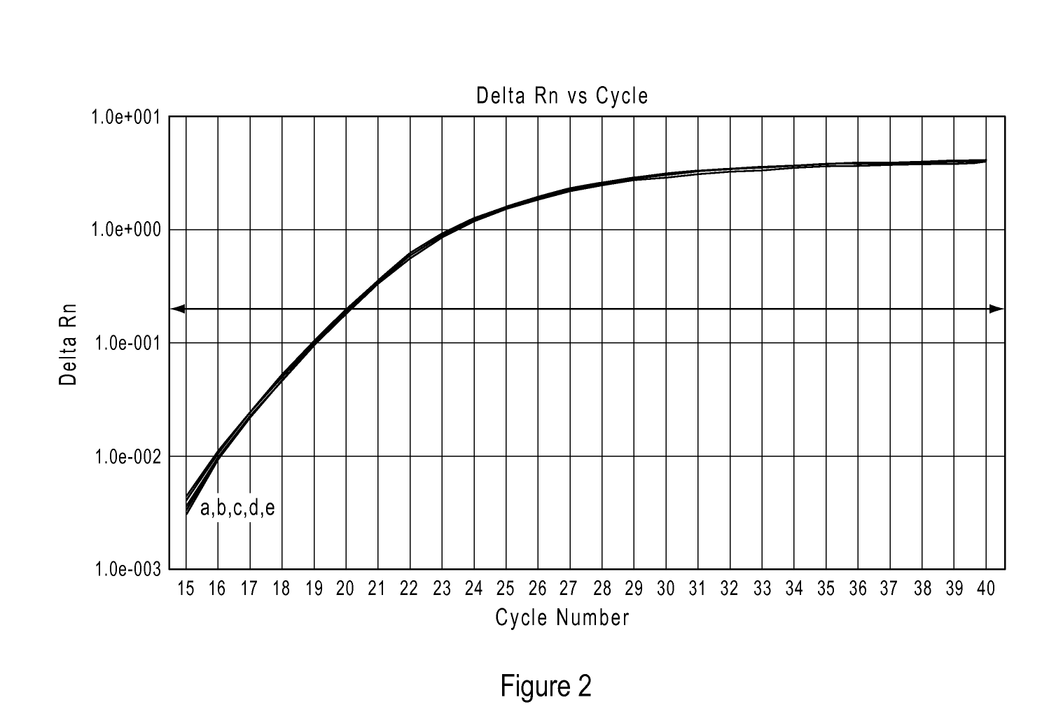

To perform the DNA digestion step, a solution containing DNase or DNase coated beads is added to the sample (exemplary samples include, but are not limited to, a component used in a reagent mixture, a reagent mixture, or an apparatus, e.g., microfuge tube) to be treated such that the solution/sample mixture contains the necessary reagents for digestion of contaminating DNA in the final sample. DNase concentrations of at least 0.005 U, at least 0.01 U, at least 0.015 U, at least 0.02 U, 0.025 U, and 0.03 U/μL or thereabouts were used to digest contaminating DNA at concentration levels of at least 10 pg, at least 15 pg, at least 20 pg, and at least 25 pg/μL. An exemplary DNase, TURBO DNase™ enzyme, at a final concentration of 0.02 U/μL digested 20 pg/μL of the contaminating DNA in the resulting mixture. The mixture is then incubated for a sufficient period of time. The greater the amount of nuclease added the shorter the length of incubation and conversely, a lower amount of nuclease added to the sample to be decontaminated, the longer the incubation period required for complete degradation of contaminating nucleases. Incubation periods can range from at least 5 to at least 60 minutes at temperatures between at least 35° C. and at least 40° C. The DNA digesting enzyme is then inactivated as would be known to one of skill in the art, including, but not limited to, by means of heat (e.g., 5 min., at at least 75° C.), or by means of separation of DNase coated beads by column filtration, centrifugation or magnetic separation. FIG. 1 illustrates the level of digestion by 0.02 U/μL of 0.2 pg/μL spiked E. coli DNA as digestion time increases. There is a 256-fold decrease in DNA after 40 min. of digestion. The inactivated nuclease does not interfere with the subsequent reactions as illustrated by FIG. 2, exemplary reactions can be PCR or Sanger sequencing. Thus, one of skill in the art would conclude that there is insignificant residual nuclease activity following inactivation or removal of the nuclease following digestion with a nuclease to be a significant factor in subsequent reactions using the nuclease treated reagent or apparatus.

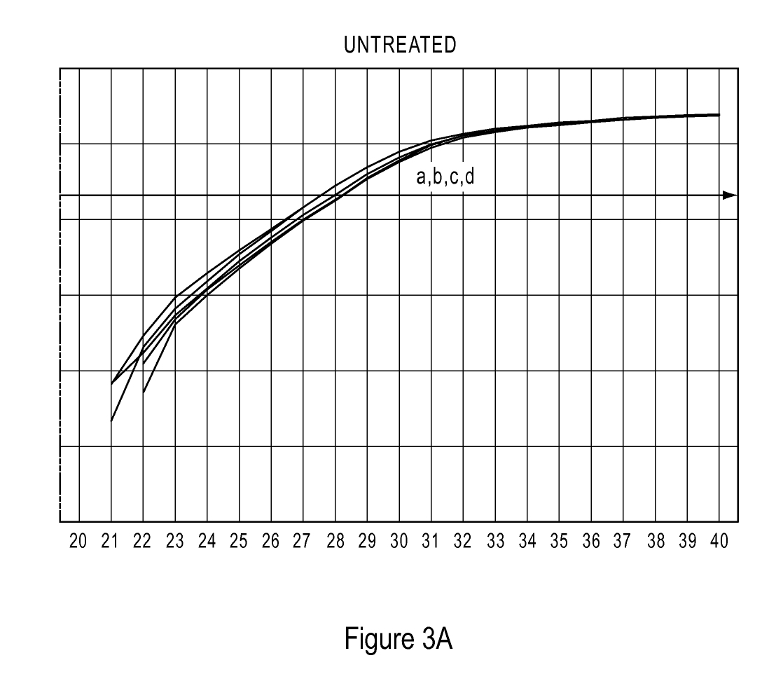

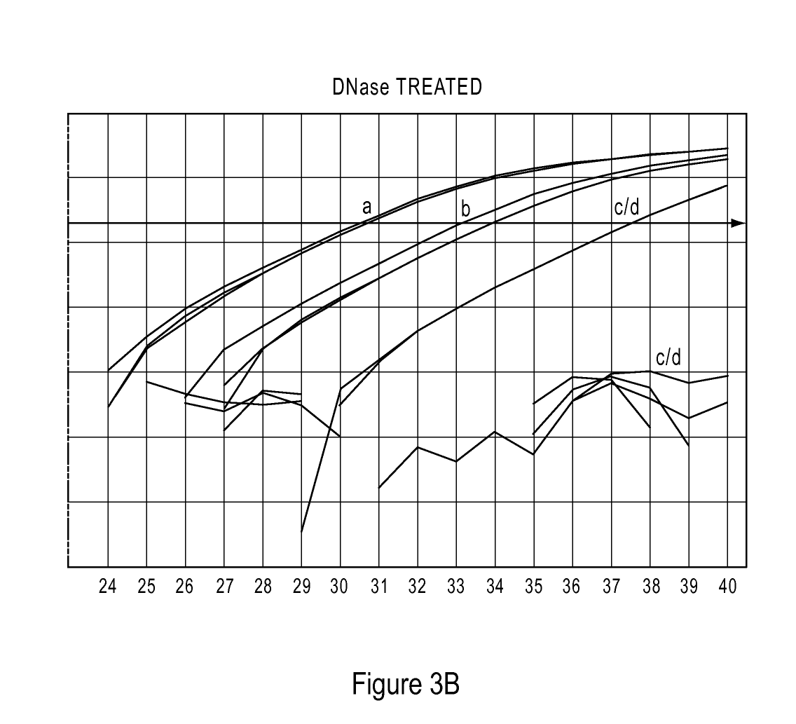

A comparison of FIGS. 1 and 3 not only indicate the sensitivity of a PCR reaction to detect pg quantities of contaminating DNA, but suggest that 20 pg/μL of DNA is sufficiently digested by 0.02 U/μL DNase after 10 min to 20 min. such that it is not a significant factor in subsequent PCR reactions. Furthermore, the digestion of 20 pg/μL of DNA using 0.0014 U/μL of DNase is also sufficient to remove contaminating DNA after a 60 min. digestion period, as seen in FIG. 3, where a single CPU is detected in the PCR reaction.

In one embodiment, the DNase, e.g., TURBO DNase™ enzyme, can be inactivated by heat after DNase treatment. Optionally, prior to DNase treatment, RNase treatment or treatment with a plurality of DNases, RNases or a combination of DNase(s) and RNase(s), a PCR master mix reagent can also be treated with ultrasonication to lyse microbes releasing their nucleic acids. PCR master mix can also be treated with heat to degrade RNase added to degrade microbial RNAs released by sonication.

In one embodiment of the present teachings of the disclosed DNA decontamination process is exposing the PCR master mix, sample prep materials (such as nucleic acid purification beads) and apparatus to DNase treatment. The reagents and materials so exposed to DNase are then treated by heat to inactivate the DNase after DNase treatment or a method to separate DNase coated beads from the decontaminated reagent. Prior to DNase treatment, PCR master mix can also be treated with ultrasonication to lyse microbes releasing their nucleic acids. PCR master mix can also be treated with heat to degrade microbial RNA.

In another embodiment, the present teachings also provide a nuclease-free: reagent, PCR master mix, a component of a PCR master mix or an apparatus used in the isolation of a nucleic acid produced by a) adding a nuclease to a contaminated reagent or apparatus, b) incubating the contaminated reagent or apparatus to digest the contaminating nucleic acid (e.g., DNA or RNA) at an effective temperature for a sufficient period of time, and c) inactivating the nuclease's activity. The nuclease can be either a DNase or an RNase, or a combination of DNases, RNases or DNase(s) plus RNase(s) added to the contaminated reagent or apparatus. The nuelease(s) may be added as a solution or bound to an insoluble matrix or a solid support, such as a bead selected from the group consisting of magnetic, non-magnetic, glass, and cellulose beads. The immobilized nuclease can be removed by filtration, centrifugation or magnetic separation. Alternatively, the nuclease is inactivated by heat following incubation. The resulting nuclease-free reagent, mixture, component or apparatus has insignificant residual nuclease activity and would not be expected to interfere in the subsequent analysis of nucleic acids by molecular biological means.

In another aspect, the present teachings provide a new method for using a DNA-digesting enzyme for removing nucleic acids from microorganisms such as E. coli. The method is effectively applicable to remove DNA from bacteria, fungi, microbes and all other biological species.

DNA-digesting enzymes can be used either as a solution or immobilized on an insoluble matrix or on a solid support. For example, PCR master mix can be passed through a column packed with immobilized DNase beads or the interior of a tube coated with DNase at an optimized flow rate and temperature to digest contaminating DNA. PCR master mix can also be mixed with DNase immobilized on beads (magnetic or non-magnetic). The treated PCR master mix can be separated from the immobilized DNase coated beads by centrifugation, filtration or by magnetic separation.

There are several advantages to using immobilized DNase or RNase in nuclease treatment methods to remove unwanted DNA and RNA, respectively. The immobilized nuclease enzymes are easily removed from a reaction mixture and consequently pose better control and rapid termination of the nuclease reaction and there is less risk of contamination of the residual nuclease enzyme in the treated reagent. The immobilized enzymes can be reused and have enhanced stability compared to free nucleases in solution.

Nucleases can be attached to solid supports using immobilization chemistries known to one of skill in the art based on the nuclease and the solid support selected. There are many known exemplary supports and methods for the attachment of nucleases including, but are not limited to, e.g., nylon and polystyrene. See, e.g., (P. Michalon, J. Roche, R. Couturier, G. Favre-Bonvin and C. Marion, Enzyme Microb. Technol. 15 (1993), p. 215-221), e.g., magnetic bead cellulose particles, see, e.g., B. Rittich, et al., I Chromatogr. B (2002) 77:25-31), e.g., SEPHAROSE, see, e.g., (A. F. M. Moorman, F. Lamie and L. A. Grivell, FEBS Lett. 71 (1976), p. 67-72.), e.g., porous glass, see, e.g., (A. R. Neurath and H. H. Weetall, FEBS Lett. (1970), 8:253-256.), e.g., convective interaction media monolithic supports, see, e.g., (M. Bencina, et al., (2008) Methods Mol Biol. 421: Affinity Chromatography: Methods and Protocols, 2^{nd} Ed. M. Zachariou, Humana Press, Totowa, p 257-274),

e.g., immobilization of DNase via epoxy groups of methacrylate supports, see, e.g., M. Bencina et al., J. Chromatography A, (2005), 1065:83-91, and e.g., polymeric brushes to immobilize RNase, see, e.g., S. P. Cullen, et al., (2008) Langmuir 24:913-920. Each reference is incorporated herein by reference in its entirety.

Filtration columns containing immobilized DNase-porous glass beads can be prepared according to the method described by Neurath et. al., (A. R. Neurath and H. H. Weetall, FEBS Lett. 8 (1970), p. 253-256). 0.25 g to 3.4 g of the DNase-glass derivative is packed into disposable chromatographic columns (catalogue No. 96010 or 96020, BioRad Laboratories, Richmond, Calif.). The column temperature is maintained at 37° C. An exemplary reagent for decontamination, e.g., PCR master mix, is recirculated through the DNase-glass derivative at speeds between 0.1 to 1.9 mL/min. to allow DNA digestion by the immobilized DNase. The PCR master mix is recovered after 60 minutes of treatment.

The use of nuclease treatment to remove DNA (e.g., from body fluids, sexual assault samples, etc.) from forensic samples is also envision. As described in Example 3, the use of DNase in the sexual assault sample is also a very different solution for removing animal nucleic acids from target samples which might otherwise interfere with the PCR reaction of a target sample, including, but not limited to a sperm DNA sample. In the case of sexual assault sample processing, DNA inside the intact cells (both sperm cells and epithelial cells) are protected from DNase digestion. Only the extraneous DNA outside the intact cells is digested.

Similarly, the use of RNase in any form is also claimed in this disclosure for removal of contaminating RNA molecules in, for example, but not limited to, a target nucleic acid sample, reagents and apparatus used in the isolation of RNA, and a PCR reaction mix and the components thereof. In one embodiment, the RNase is immobilized on a solid support, including, but not limited to, an insoluble matrix, a column, a bead, a tube, and so on and separated from the decontaminated solution after RNA digestion by filtration, centrifugation, magnetic separation, and so on.

The components of the PCR master mix can be adjusted and varied according to the compatibility or the design of the assay as would be understood by one of skill in the art.

The methods described herein can also be used in conjunction with other techniques such as filtration and magnetic separation to achieve the goal of making nucleic acid-free reagents, materials and apparatus.

### EXAMPLES

The following procedures are representative of procedures that can be employed for the enzymatic removal of nucleic acids from reagents, materials and apparatus used in molecular biological, diagnostic and pharmaceutical research as well as molecular biological and biopharmaceutical manufacturing applications.

### Example 1

### Demonstration of TURBO DNase™ Enzyme Activity in PCR Master Mix

E. coli DNA was added to PCR master mix (not including primers or probe(s)) for a final DNA concentration of 20 pg/μL in the PCR reaction. TURBO DNase™ enzyme (Ambion) (final concentration of 0.02 U/μL was then added to the PCR reaction mix and placed in a heat block set at 37° C. for various digestion times as shown in Table 2.

TABLE 2

| | reaction | | reaction | | reaction | | reaction | | reaction | |
|---|---|---|---|---|---|---|---|---|---|---|
| Reagent | a | | b | | c | | d | | e | |
| E. coli DNA | 4 | uL | 4 | uL | 4 | uL | 4 | uL | 4 | uL |
| (100 pg/uL) | | | | | | | | | | |
| TaqMan ® | 10 | uL | 10 | uL | 10 | uL | 10 | uL | 10 | uL |
| Gene | | | | | | | | | | |
| Expression | | | | | | | | | | |
| Master | | | | | | | | | | |
| Mix (PN: | | | | | | | | | | |
| 4389986) | | | | | | | | | | |
| water | 3 | uL | 3 | uL | 3 | uL | 3 | uL | 3 | uL |
| Turbo | 1 | uL | 1 | uL | 1 | uL | 1 | uL | 1 | uL |
| DNase ™ | | | | | | | | | | |
| (0.02 U/uL) | | | | | | | | | | |
| DNase | 0 | min | 10 | min | 20 | min | 30 | min | 40 | min |
| digestion | | | | | | | | | | |
| at 37° C. | | | | | | | | | | |
| DNase heat | 10 | min | 10 | min | 10 | min | 10 | min | 10 | min |
| inactivation | | | | | | | | | | |
| at 75° C. | | | | | | | | | | |
| forward | 1 | uL | 1 | uL | 1 | uL | 1 | uL | 1 | uL |
| Primer (20x) | | | | | | | | | | |
| reverse | 1 | uL | 1 | uL | 1 | uL | 1 | uL | 1 | uL |
| Primer (20x) | | | | | | | | | | |

Aliquots of the PCR master mix/DNase mixture (15 μL) were removed at times 0 min., 10 min., 20 min., 30 min. and 40 min. after DNase digestion was initiated. Each aliquot was placed in a heat block set at 75° C. for 10 min. to inactivate the TURBO DNase™ enzyme. Following inactivation, 1 μL forward primer, 1 μL reverse primer and 34 of water were added to each DNase treated PCR master mix aliquots. Each aliquot of TURBO DNase™ enzyme treated PCR master mix from the various time points were tested in triplicate by PCR to determine how much of the spiked E. coli DNA has been digested. The PCR conditions were 10 minutes incubation at 95° C., then 40 cycles between 95° C. (15 seconds) and 60° C. (1 minutes), followed by a dissociation stage (15 second at 95° C., 1 minute at 60° C. and 15 minute at 95° C.). As seen in FIG. 1, the residual DNA amount decreases (indicated by increasing in C_{T} value) with increased DNase digestion time. Therefore, TURBO DNase enzyme is still active in the reagent mixture that constitutes the PCR master mix. The C_{T} value increased by about 8 (Delta Rxn. Vs. Cycle), after 40 min. of TURBO DNase™ enzyme digestion, which indicates a 256-fold reduction in added DNA as a result of the TURBO DNase™ enzyme treatment.
The E. coli DNA amount decreases with increased DNase digestion time.

FIG. 2 illustrates the effectiveness of inactivation of TURBO DNase™ enzyme by heat. The enzyme was first heated at 75° C. for 10 min. before being used for DNA digestion in PCR reaction mixes. No C_{T} shifts and thus, no DNA digestion, even after 40 minutes of digestion at 37° C. were observed This demonstrates the ability to inactivate the DNase by heating, thus, preserving the primers and probe(s) (if a real-time PCR reaction) and target sample nucleic acid, preventing their degradation when added to the decontaminated, nuclease-inactivated PCR master mix.

### Example 2

### PCR Master Mix Decontamination by TURBO DNase™ Enzyme and the Use of Decontaminated PCR Master Mix for Detection of Trace Amounts of E. Coli DNA

To demonstrate the effectiveness of DNase treatment for the removal of contaminating DNA in a PCR master mix, DNase treated PCR master mix was used for the detection of trace amounts of E. coli DNA.

TURBO DNase™ enzyme was added to PCR master mix (not including primers and probes) as described in Example 1 (the reaction mixture was: 1 uL of DNase (0.2 U/uL) added to 10 uL of PCR master mix and 3 uL of water. The final DNase reaction volume was 14 μL) having a final DNase concentration of 0.0014 U/μL. DNA digestion was carried out at 37° C. for 60 min. followed by 75° C. incubation for 10 min. to inactivate TURBO DNase™ enzyme activity. The DNase treated PCR master mix was then used for detection in triplicate, of 100 copies, 10 copies, 1 copy and 0 copies of E. coli genomic DNA and the results were compared to that obtained with untreated PCR master mix.

Untreated PCR master mix used for targeting E. coli DNA by PCR amplification yielding C_{T} values of 28 for all E. coli concentrations, including the no template control (NTC) (See FIG. 3, left). If there was no residual E. coli DNA contamination, than the C_{T} for the NTC should be greater than 40. A C_{T} for the NTC of 28 clearly indicates bacterial DNA contamination in the PCR master mix. Since the C_{T} value for 100 copies of E. coli is also 28, the contamination level in the NTC is indicative of at least 100 copies contaminating E. coli DNA and detection of contaminating nucleic acid of less than 100 copies cannot be achieved when using an untreated PCR master mix. In FIG. 3 the y-axis is Delta Rn and the x-axis is C_{T}.

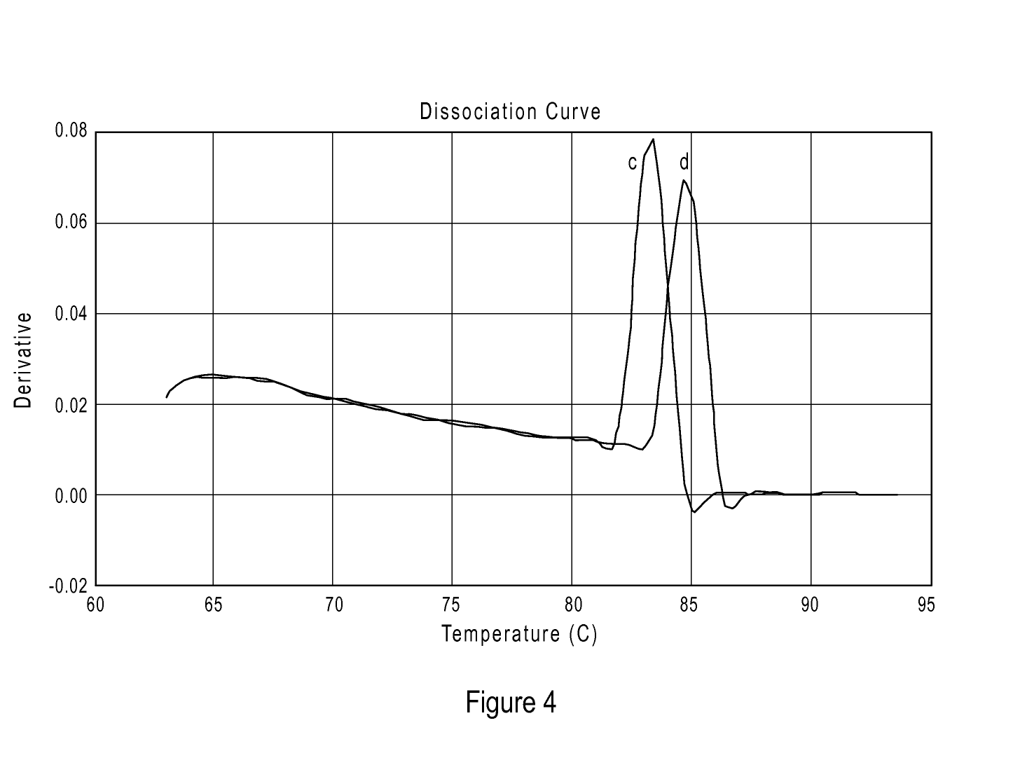

TURBO DNase™ enzyme treated PCR master mix resulted in C_{T} values of about 30.6 and 33.5 for the three 100 copy reactions and the three 10 copy reactions, respectively. One of the three 1 copy reactions gave a C_{T} of 37.5 while the other two reactions failed to amplify. This is probably due to the stochastic effect or an absence of target DNA in the two failed reactions. Of the three NTC reactions, two of the reaction also did not amplify. The one NTC amplified reaction gave a C_{T} value of 37.5, but the melting curve of this amplified NTC product is different from that of the E. coli amplicon (see FIG. 4). Therefore, using a DNase enzyme treated PCR master mix, detection of a single copy of a DNA target was achieved. As shown in FIG. 3 right, a single copy of an E. coli DNA target was detected following DNase treatment of the PCR master mix. The use of untreated PCR master mix looses sensitivity as the contamination of exogenous DNA is so high that even 100 copies of spiked E. coli DNA can not be differentiated from NTC (FIG. 3, left).

### Example 3

### DNase Treatment of a Sexual Assault Sample to Isolate Sperm DNA

To a 50 μL sexual assault sample (the sample can have sperm cells, epithelial cells, blood cells and extraneous DNA)s, 50 μL of a cell wash solution is added to a 1.5 mL tube. The solutions are mixed and incubated at room temperature 25° C. for 5 min. This process removes extraneous DNA and lyses any blood cells, if present. The sample is then centrifuges at 14K rpm for 1 min. to pellet the cells, the supernatant is carefully removed and discarded. The pellet can have sperm cells and epithelial cells and extraneous DNA which can stick to the surface of these cells. 200 μL of Danes lysis buffer and 2 μL TURBO DNase™ is added to the cell pellet which is then mixed and incubated at 37° C. for 5 min. This process allows any extraneous DNA external to the cells to be digested. Because the DNase can not penetrate the cell membrane the DNA inside sperm cells and epithelial cells will not be digested. The mixture is again centrifuged at 14K rpm for 1 min. to pellet the sperm and epithelial cells. The supernatant is carefully removed from the pellet and discarded, leaving a pellet free of extraneous DNA (the supernatant contained the DNase too). The pellet is re-suspended in 200 μL selective sperm lysis reagent, mixed and incubate for 5 min. at room temperature (25° C.) The selective sperm lysis reagent selectively lyse sperm cells, releasing sperm DNA while leaving epithelial cells intact. The isolate sperm DNA can then be used in PCR methods to identify the source of the sperm DNA.

All publications and patents, mentioned in the above specification are herein incorporated by references. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments.

While the foregoing specification teaches the principles of the present invention, with examples provided for the purpose of illustration, it will be appreciated by one skilled in the art from reading this disclosure that various changes in form and detail can be made without departing from the spirit and scope of the invention. Indeed, various modifications of the above-described modes for carrying out the invention, which are obvious to those skilled in the field of protein chemistry, molecular biology or related fields are intended to be within the scope of the following claims.

1. A method for removing nucleic acid contamination in a PCR master mix reagent comprising:
a. adding a nuclease to the PCR master mix reagent;
b. incubating the PCR master mix reagent plus nuclease; and
c. inactivating the nuclease.

2. The method of claim 1, wherein the PCR master mix is formulated for amplifying microorganism nucleic acid.

3. The method of claim 1, wherein the nuclease is a DNase or an RNase.

4. The method of claim 1, wherein the inactivation of the nuclease is by heat.

5-7. (canceled)

8. The method of claim 6, further comprising removal of the nuclease by centrifugation, filtration or magnetic separation.

9. A method for removing DNA contamination in a solution comprising:
a. passing the solution through a bead, an insoluble matrix, or through a tube, wherein the bead, the insoluble matrix, or the internal surface of said tube comprises at least one DNase; and
b. collecting a DNase treated solution from the bead, the insoluble matrix or the tube.

10. A method for removing RNA contamination in a solution comprising:
a. adding RNase to the solution;
b. incubating the solution with RNase; and
c. inactivating the RNase.

11. The method of claim 10, wherein the RNase is bound to a solid surface.

12. The method of claim 11, wherein the solid surface is a bead.

13. The method of claim 9, wherein the bead is selected from a group consisting magnetic, non-magnetic, glass and cellulose beads.

14-22. (canceled)

23. The method of claim 9 wherein said bead or said insoluble matrix is in a column.

24. The method of claim 9 wherein said DNase is coated or immobilized on the bead or the insoluble matrix.

25. The method of claim 9 wherein the DNase treated solution is incubated at about 75° C. to produce a resultant solution.

26. The method of claim 9 wherein the DNase is selected from the group consisting of natural, synthetic or chemically modified DNase.

27. The method of claim 25 wherein the resultant solution is substantially free of residual DNase activity.

28. The method of claim 9 wherein the DNase is a recombinant DNase I.

29. The method of claim 28 wherein the natural DNase is of prokaryotic or eukaryotic origin.

30. The method of claim 28 wherein the recombinant DNase is TURBO™.

31. The method of claim 29 wherein the DNase is derived from an organism of either mammalian, nematode, bacterial, yeast, fungal, plant or marine origin.
